(19) United States
(12) Patent Application Publication
(10) Pub. No.: US 2025/0213150 A1
(43) Pub. Date: Jul. 3, 2025

(54) SENSING SYSTEM AND METHOD USING GALVANIC SEPARATION

(71) Applicant: QuLab Medical Ltd., Herzliya (IL)

(72) Inventors: Sharon Lefler, Herzliya (IL); Idan Tamir, Herzliya (IL); Hila Masasa, Herzliya (IL); Berta Ben-Shachar, Herzliya (IL)

(21) Appl. No.: 18/881,167

(22) PCT Filed: Jan. 13, 2023
(86) PCT No.: PCT/IB2023/000017

§ 371 Date: Jan. 3, 2025

(30) Foreign Application Priority Data
Jan. 13, 2022 (US) 63299308

(51) Int. Cl. A61B 5/1473; A61B 5/00

(52) U.S. Cl. CPC A61B 5/1473; A61B 5/685; A61B 2560/0204

(57) ABSTRACT

A sensor system including a plurality of potentiometric sensors, wherein each of the potentiometric sensors has a sensor area of less than 1 square centimeter, wherein each of the potentiometric sensors comprises one working electrode, and wherein each one of the potentiometric sensors is galvanically separated from all other ones of the potentiometric sensors so as to avoid measurement crosstalk among the plurality of potentiometric sensors; a plurality of sensor control subunits, wherein each of the sensor control subunits is communicatively coupled to a particular corresponding one of the plurality of potentiometric sensors, and wherein each one of the sensor control subunits is galvanically separated from all other ones of the sensor control subunits so as to avoid measurement crosstalk among the plurality of sensor control subunits; and a main control unit communicatively coupled to each of the plurality of sensor control subunits.

### CROSS-REFERENCE TO RELATED APPLICATION

This application is a national phase filing under 35 U.S.C. 371 of International (PCT) Patent Application No. PCT/IB2023/000017, filed on Jan. 13, 2023, and entitled “SENSING SYSTEM AND METHOD USING GALVANIC SEPARATION,” which claims the benefit of commonly-owned, U.S. Provisional Patent Application No. 63/299,308, filed on Jan. 13, 2022, and entitled “SYSTEM AND METHOD FOR CONTINUOUS MULTI-PARAMETER RECORDING USING GALVANIC ISOLATED CIRCUITS,” the contents of which are incorporated herein by reference in their entirety.

### FIELD

The present disclosure relates to a sensor device including a plurality of sensor microprobes.

### BACKGROUND

Bio-analyte sensing using microprobes has the advantage of minimal invasiveness. Micro-sensing systems, such as sensors mounted on microneedles, microprobes or neural probes are commonly used for healthcare applications (among other applications). The minimal invasive approach has the dual advantage of inflicting less pain and being less prone to infections. However, sensors that are positioned at microscopic distances from one another may experience crosstalk that may impact the quality of data captured by such sensors.

### SUMMARY OF THE INVENTION

This summary is a high-level overview of various aspects of the invention and introduces some of the concepts that are further detailed in the Detailed Description section below. This summary is not intended to identify key or essential features of the claimed subject matter, nor is it intended to be used in isolation to determine the scope of the claimed subject matter. The subject matter should be understood by reference to the appropriate portions of the entire specification, any or all drawings, and each claim.

In some embodiments, a sensor system includes a plurality of potentiometric sensors, wherein each of the potentiometric sensors has a sensor area of less than 1 square centimeter, wherein each of the potentiometric sensors includes one working electrode, wherein each one of the potentiometric sensors is galvanically separated from all other ones of the potentiometric sensors so as to avoid measurement crosstalk among the plurality of potentiometric sensors; a plurality of sensor control subunits, wherein each of the sensor control subunits is communicatively coupled to a particular corresponding one of the plurality of potentiometric sensors, and wherein each one of the sensor control subunits is galvanically separated from all other ones of the sensor control subunits so as to avoid measurement crosstalk among the plurality of sensor control subunits; and a main control unit communicatively coupled to each of the plurality of sensor control subunits.

In some embodiments, the potentiometric sensors and the sensor control subunits are galvanically separated from one another by physical separation that no electrical or dielectric contact is present between the potentiometric sensors and the sensor control subunits. In some embodiments, the physical separation is achieved by physical bulk separation at wafer level. In some embodiments, physical separation is achieved by a dielectric or polymeric material.

In some embodiments, the main control unit and the plurality of sensor control subunits are galvanically separated from one another through use of wireless or capacitance power transmission.

In some embodiments, the main control unit and the plurality of sensor control subunits communicate with one another while galvanically separated by wireless, capacitance, optical or RF data signal transfer

In some embodiments, the main control unit is connected to the plurality of sensor control subunits by a switch that is configured to provide active connection between the main control unit and only one of the plurality of sensor control subunits at any point in time. In some embodiments, each of the plurality of sensor control subunits includes an internal power storage, and wherein the internal power storage powers the sensor control subunits when not actively connected to the main control unit.

In some embodiments, wherein the main control unit and each of the sensor control subunits each include a microcontroller.

In some embodiments, the main control unit includes a microcontroller, and wherein each of the sensor control subunits does not include a microcontroller.

In some embodiments, each of the sensor control subunits includes an analog front end. In some embodiments, the analog front end includes an application-specific integrated circuit.

In some embodiments, the main control unit and the plurality of sensor control subunits are implemented on a printed circuit board.

In some embodiments, a support layer is positioned between (1) the printed circuit board and (2) the main control unit and the plurality of sensor control subunits. In some embodiments, the main control unit and the plurality of sensor control subunits are fixed to the support layer by a non-dielectric adhesive.

In some embodiments, each of the potentiometric sensors includes only one working electrode.

In some embodiments, each of the potentiometric sensors includes no reference electrode.

### BRIEF DESCRIPTION OF THE DRAWINGS

Some embodiments of the present disclosure are herein described, by way of example only, with reference to the accompanying drawings. With specific reference now to the drawings in detail, particulars shown are by way of example and for purposes of illustrative discussion of some embodiments of the invention. In this regard, the description taken with the drawings makes apparent to those skilled in the art how embodiments of the invention may be practiced.

FIG. 1A shows an exemplary embodiment of a sensor array.

FIG. 1B shows an exemplary embodiment of multiple sensors.

FIG. 2A shows an illustration of an exemplary wafer-level stack configuration of a multi-layered assembly structure.

FIG. 2B shows an exemplary sensor array.

FIG. 3A shows an exemplary sheet of material for forming a support layer.

FIG. 3B shows two exemplary sensors bonded to a support layer.

FIG. 4 shows a circuit diagram for an exemplary sensor device.

FIG. 5 shows a circuit diagram for an exemplary sensor device.

FIG. 6 shows a circuit diagram for an exemplary sensor device.

FIG. 7A shows a diagram illustrating the operation of virtual floating voltage control.

FIG. 7B shows a diagram illustrating the operation of virtual floating voltage control.

FIG. 7C shows a diagram illustrating the operation of virtual floating voltage control.

FIG. 8 shows an exemplary embodiment of a sensing device.

FIG. 9 shows an exemplary embodiment of a sensing device.

FIG. 10 shows an exemplary embodiment of a sensing device.

FIG. 11 shows an exemplary embodiment of a sensing device.

FIG. 12 shows an exemplary embodiment of a sensing device.

### DETAILED DESCRIPTION

The following description of the preferred embodiment(s) is merely exemplary in nature and is in no way intended to limit the invention, its application, or uses. As used throughout, ranges are used as shorthand for describing each and every value that is within the range. Any value within the range can be selected as the terminus of the range. In addition, all references cited herein are hereby incorporated by referenced in their entireties. In the event of a conflict between a definition in the present disclosure and that of a cited reference, the present disclosure prevails.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention.

Relative terms such as “lower,” “upper,” “horizontal,” “vertical,” “above,” “below,” “up,” “down,” “left,” “right,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such.

Terms such as “attached,” “affixed,” “connected,” “coupled,” “interconnected,” “mounted” and similar refer to a relationship wherein structures are secured or attached to one another either directly or indirectly through intervening structures, as well as both movable or rigid attachments or relationships, unless expressly described otherwise.

As used in the specification and the claims, the singular form of “a”, “an”, and “the” include plural referents unless the context clearly dictates otherwise.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The term “about” means a range of plus or minus ten percent of the stated value.

Unless otherwise indicated, all ranges or ratios disclosed herein are to be understood to encompass any and all subranges or subratios subsumed therein. For example, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 6.1, 3.5 to 7.8, and 5.5 to 10.

The terms “first”, “second”, and the like are not intended to refer to any particular order or chronology, but instead refer to different conditions, properties, or elements.

All documents referred to herein are “incorporated by reference” in their entirety.

The term “at least” means “greater than or equal to”. The term “not greater than” means “less than or equal to”.

As used herein, the term “microprobe” is interchangeable with the terms “microneedle” and “neural probe”.

As used herein, the term “distal end” is defined as a distal-most point or line of a unit. For example, the distal end of a tip of a microprobe is the part of the microprobe that contacts the skin first.

The exemplary embodiments relate, but are not limited, to miniaturized, solid state potentiometric sensors that employ only one working electrode. Examples of such sensors are described in International Patent Application Publication No. WO2021/009559, titled ELECTROCHEMICAL FET SENSOR, and in International Patent Application Publication No. WO2021/144651, titled SENSING SYSTEM INCLUDING LAYERED MICROPROBE, the contents of both of which are incorporated herein by reference in their entirety. In some embodiments, such sensors exploit the high sensitivity of a silicon nanowire field effect transistor (SiNW FET) to detect the changes in working electrode potentials driven by interaction of redox species or ions with the electrode surface. In some embodiments, such sensors have a very small sensor area (e.g., about 50 μm×50 μm), and such very small sensors, as implemented through the use of a dedicated electronic transducer, allow for multi-metabolite sensing and background signal subtraction. However, the inventors of the present application have discovered that recording several analytes by closely-positioned potentiometric sensors (e.g., within a few centimeters of one another), and having the same electrical ground, potentially causes crosstalk to occur between the different sensors. Potentiometric sensors measure small changes in the working electrode potential utilizing a sensitive field effect transistor situated in close proximity to the working electrode for signal amplification. Some such sensing systems do not include a reference electrode to which any given sensor signal can be differentially referenced. Thus, in the case of a mixed signal resulting from two or more different working electrodes, the coupled field effect transistor may sense an average/mixed potential change for all the separate working electrodes.

The exemplary embodiments described herein relate to electrical decoupling of the different field effect transistor-based sensors, including their working electrodes and backgates. In other embodiments, similar platforms and methods using galvanic separation are applicable to other types of multi-sensor arrays which display signal crosstalk. In some embodiments, such decoupling separates the signals of different sensors of a single sensor device, even when such sensors are in close proximity to one another (e.g., within one millimeter of one another) within a liquid medium. In some embodiments, galvanic isolation mainly blocks electrical currents between two differentially grounded electrical circuits. In some embodiments, total wireless galvanic separation, as discussed herein, also decouples (e.g., completely decouples) relative potential interaction that may interfere with potentiometric based sensors. In some embodiments, to avoid sensor crosstalk among sensor arrays in a common buffer/solution, and at close proximity in non-referenced sensing setups, galvanic separation (e.g., complete galvanic separation) is utilized.

In some embodiments, different sensors, such as field effect transistor-based sensors, that are positioned on the same conductive or dielectric substrate may suffer from potential interference, and specifically if their respective gates are not positioned in proximity (e.g., under 1 μm) to the FET channel of the corresponding sensor. In some embodiments, inserting a non-dielectric isolation, such as an air gap or a polymer, between sensors can dramatically decrease sensor-to-sensor interference. In some embodiments, crosstalk prevention enables simultaneous recording of multiple parameters by different sensors within a sensor array. In some embodiments, analog or digital galvanic separation can be achieved in accordance with one of the embodiments described hereinafter.

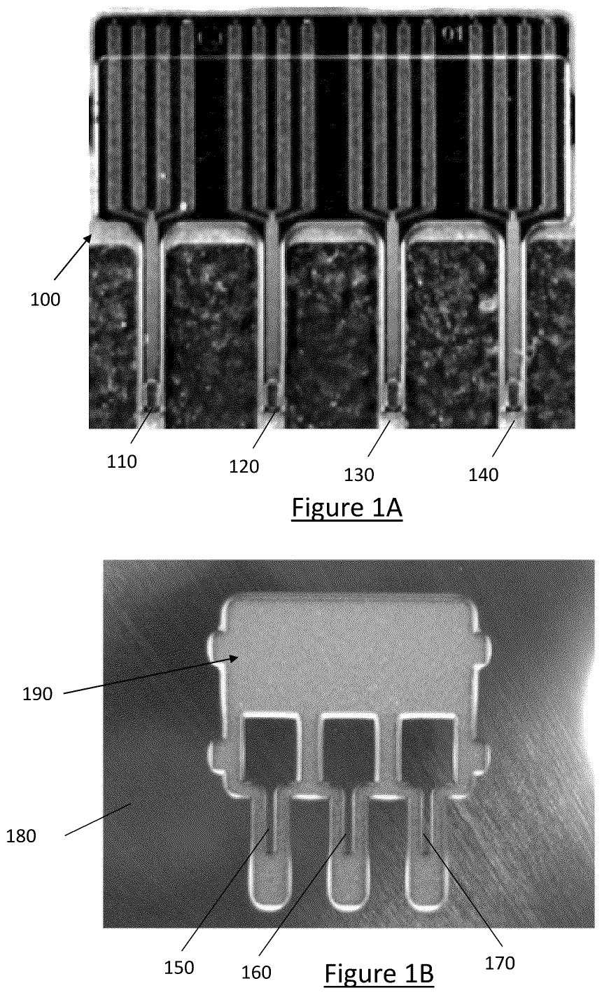

In some embodiments, a silicon-on-insulator (SOI) substrate includes a relatively thin (e.g., having a thickness on the order of 100 nanometers to several micrometers) buried oxide dielectric insulation layer. In some embodiments, such a buried oxide dielectric insulator allows the propagation of electric potential over the buried oxide layer, which hinders electric potential galvanic separation. FIG. 1A shows an exemplary embodiment of a sensor array 100 including multiple sensors 110, 120, 130, 140 that are formed on a single substrate. In some embodiments, to provide sufficient galvanic separation to prevent crosstalk, conductive elements are separated from a dielectric layer to thereby achieve sufficient galvanic separation. FIG. 1B shows an exemplary embodiment of multiple sensors 150, 160, 170 that are galvanically separated (e.g., by air gaps). As shown in FIG. 1B, the sensors 150, 160, 170 have been singulated from a wafer 180 and are shown on an underlying tape 190 that retains the sensors 150, 160, 170 in a fixed position relative to one another for assembly into a sensor array as described herein. In the embodiment shown in FIG. 1B, the sensors 150, 160, 170 have been grained to 50 micrometers. Moreover, in some embodiments, a different backgate biasing voltage is applied to each sensor within a sensor array in order to change the transistor characteristics of each sensor and to thereby modify the electrochemical properties thereof. In some embodiments, physical singulation of the substrate underlying the backgate of each sensor (e.g., galvanic separation of each backgate) allows for the application of such independent sensor-specific voltage. In some embodiments, galvanic separation of sensor probes can also be achieved using thicker dielectrics (e.g., having a thickness on the order of over 100 micrometers), such as silicon oxide or other oxides, or by the inclusion of thin (e.g., on the order of greater than 0.1 nanometers) non-dielectric materials, such non-dielectric polymers such as polyimides or SU-8 photoresists, which will provide for the gaps in-between the probes.

In some embodiments, to achieve physical separation (e.g., galvanic separation) of adjacent sensors while maintaining constant relative positions of adjacent sensors, sensors are positioned on a support substrate (e.g., a hard or flexible support substrate) in a wafer-level setup. In some embodiments, the support layer includes a non-dielectric material, a dielectric material, or a metal, and is insulated from the sensor itself by a polymeric and non-dielectric adhesive. For example, in some embodiments, the sensor chip is positioned on a dielectric or conductive support substrate that is precoated with a non-dielectric layer, such as a polymeric adhesive. In some embodiments, any number of sensors (e.g., two sensors, three sensors, or more sensors) are physically isolated from one another (e.g., galvanically separated from one another) and mounted onto a common support layer using a polymeric and non-dielectric adhesive layer. In some embodiments, a fabrication process includes depositing an adhesive (e.g., a polymeric and non-dielectric adhesive) on a support substrate at the wafer level, bonding (by the deposited adhesive) the support substrate to an etched and singulated silicon wafer containing the sensors, and cutting the support substrate. In some embodiments, the support layer is cut using a laser or other mechanical or chemical cutting process. In some embodiments, to provide for electrical connectivity, a wafer-level printed circuit board is bonded under the support layer such that laser-cut slots allow for direct wire bonding of the silicon chip to the printed circuit board through these slots.

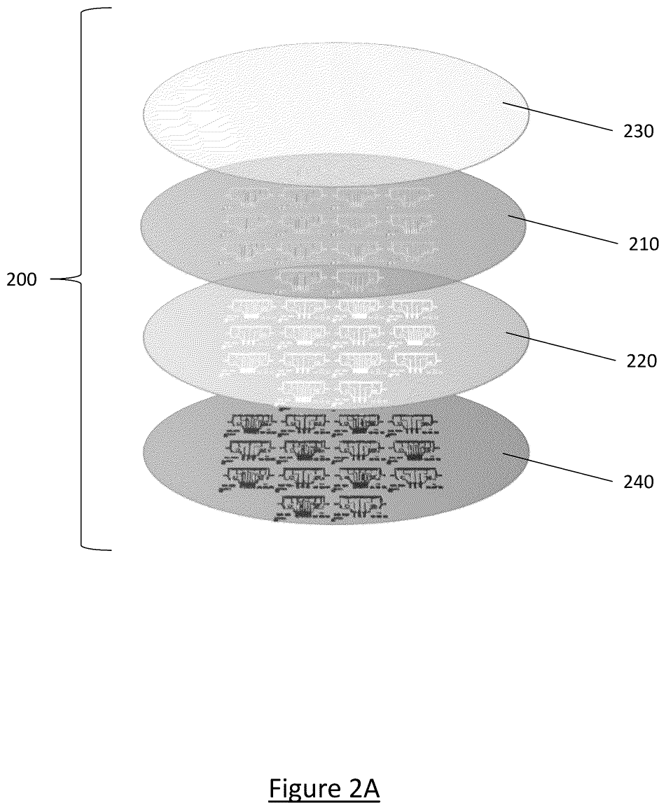

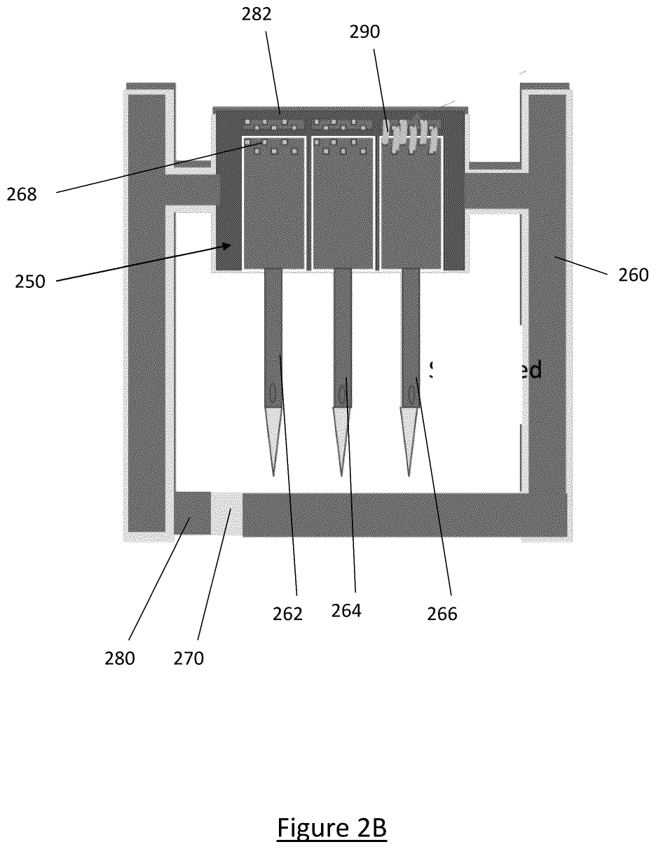

FIG. 2A shows an illustration of wafer-level stack configuration of multi-layered assembly structure as described above. The structure 200 shown in FIG. 2A includes a complementary metal-oxide semiconductor (CMOS) based silicon wafer 210. In the embodiment shown in FIG. 2A, the wafer 210 is bonded to a support layer 220. In some embodiments, the support layer 220 comprises a metal. In some embodiments, the support layer 220 comprises stainless steel. In some embodiments, the support layer 220 comprises another flexible or rigid material capable of mechanically supporting the wafer 210. In some embodiments, an adhesive (e.g., a polymeric and non-dielectric adhesive) bonds the wafer 210 to the support layer 220 as described above. FIG. 2A shows an adhesive tape 230 that is used to retain the wafer 210 in order to transfer the wafer 210 onto the support layer 220. As shown in FIG. 2A, the dual-layer structure (e.g., including the wafer 210 and the support layer 220) is then mounted to a wafer-level printed circuit board 240. In some embodiments, mounting to the wafer-level printed circuit board is accomplished using an adhesive.

FIG. 2B shows a sensor array 250. In some embodiments, the sensor array 250 includes a wafer layer (e.g., analogous to the wafer 210 described above) that includes three sensors 262, 264, 266 that are galvanically separated from one another prior to singulation. In the embodiment shown in FIG. 2B, the sensor array 250 includes a support layer 270 (shown in cutaway, e.g., analogous to the support layer 220) that includes slots to allow wire bonding of the wafer 260 (e.g., including the sensors 262, 264, 266) to a bottom-positioned printed circuit board 280 (e.g., analogous to the printed circuit board 240). In some embodiments, the printed circuit board 280 includes bond pads 282 positioned on the upper side of the printed circuit board 280 (e.g., on the side facing the support layer 270) and facing the opening in the support layer 270. In some embodiments, the wafer 260 includes connectors 268 facing downward (e.g., toward the support layer 270). In some embodiments, by connecting the connectors 268 to the bond pads 282, such as by bond wires 290 (shown only for the sensor 264 for clarity), the sensors 262, 264, 266 that are formed from the wafer 260 are able to communicate via the printed circuit board 280 and to an electronics readout system after the sensor array 250 has been singulated.

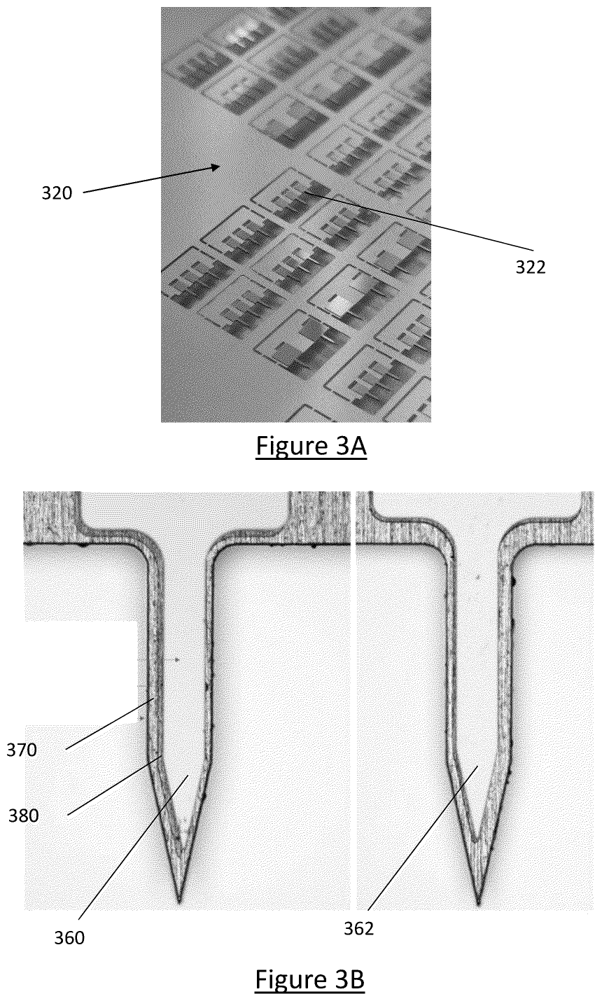

FIG. 3A shows a sheet 320 of material for forming a support layer such as the support layer 220 described above. In the embodiment shown in FIG. 3A, the material is stainless steel; as described above, in other embodiments, other materials may be used. As shown in FIG. 3A, the sheet 320 has been cut (e.g., laser cut) into several portions 322 (only one of the portions 322 specifically identified in FIG. 3B for clarity), wherein each of the portions 322 is sized and shaped to form the support layer 220 for a single sensor array.

FIG. 3B shows two physically singulated silicon wafer-based sensors 360, 362 (e.g., sensors fabricated as described above with reference to FIG. 1B) bonded to a support layer 370 using an adhesive 380. In the embodiment shown in FIG. 3B, the support layer 370 comprises stainless steel; as described above, in other embodiments, the support layer may comprise another suitable material. In the embodiment shown in FIG. 3B, the adhesive 380 is a non-dielectric polymeric adhesive; in other embodiments, the adhesive may comprise another suitable non-dielectric adhesive.

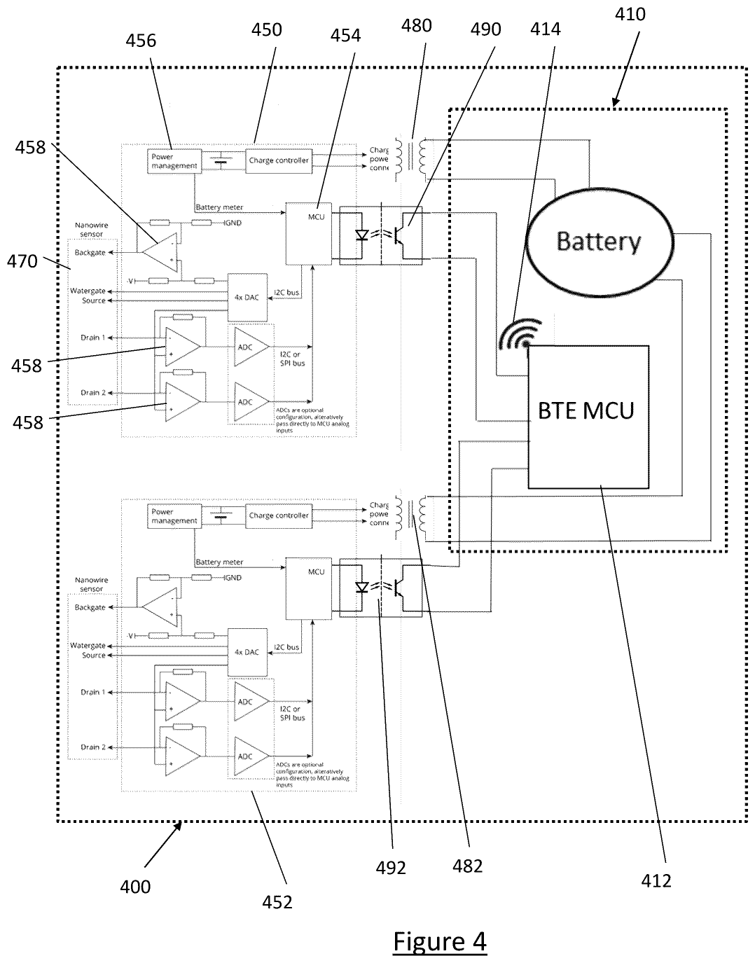

Various embodiments described herein may employ different electronic setups in order to provide galvanic separation among different sensors, to thereby prevent crosstalk as discussed herein. In some embodiments, galvanic separation is provided using an induction power supply or other wireless power transfer connection, and data (e.g., in either analog or digital format) is transferred using opto-isolators, RF transmission, induction, capacitors or other wireless data transfer connection. FIG. 4 shows a circuit diagram for such a device 400 including a main module (e.g., a main control unit) 410 and two sub-modules (e.g., sensor control subunits) 450, 452. In some embodiments, each of the sub-modules 450, 452 includes a microcontroller controller unit (MCU) 454, a power management element 456, and one or more amplifiers 458 (reference numerals only shown for one of the sub-modules 450 for clarity). In some embodiments, each of the sub-modules 450, 452 includes additional electronic hardware elements such as a multiplexer, a memory, etc. In some embodiments, the one or more amplifiers 458 are coupled to a sensor 470 (e.g., having a physically separated sensing element as described above). In some embodiments, each of the sub-modules 450, 452 is powered by a corresponding power supply apparatus 480, 482, which are galvanically separated from one another. In some embodiments, such as shown in FIG. 4, the power supply apparatuses 480, 482 are wireless inductance power supplies. In some embodiments, the sub-modules 450, 452 are connected to the main module 410 under galvanic separation conditions. In some embodiments, the sub-modules 450, 452 are wirelessly connected to the main module 410. In some embodiments, the main module 410 includes an MCU 412 and a communications module 414. In some embodiments, the sub-modules 450, 452 are digitally connected to the main module 410 by galvanically separated signal transducers 490, 492. In some embodiments, the galvanically separated signal transducers 490, 492 include an opto-isolator or other galvanic signal separation device such as capacitors, Hall effect sensors, magneto resistors, RF, transformers, or relays. In some embodiment, the main module 410 comprises only an MCU, while the sub-modules 450, 452 comprise only analog components. In the embodiment shown in FIG. 4, the device 400 includes two sub-modules 450, 452 and two sensors 470; it will be apparent to those of skill in the art that this quantity is only exemplary and that other embodiments may include other quantities of sub-modules and sensors.

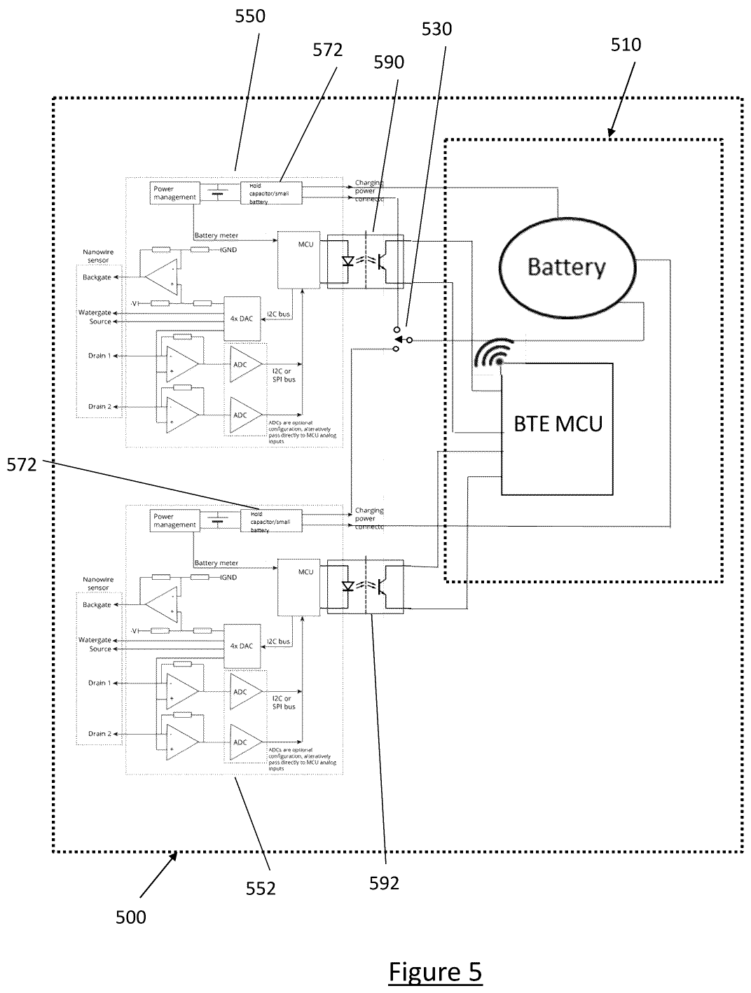

In some embodiments, galvanic separation is provided using temporal isolation. FIG. 5 shows a circuit diagram for a device 500 providing galvanic separation using temporal isolation. In some embodiments, the device 500 is generally similar to the device 400 described above other than as will be described hereinafter. In some embodiments, the device 500 includes two sub-modules 550, 552 that are connected to a main module 510. In some embodiments, the sub-modules 550, 552 are sequentially connected to the main module 510 using a galvanic switching system 530 such that only one of the sub-modules 550, 552 has active connection with the main module 510 at any point in time. In some embodiments, each sub-module 550, 552 includes a switched power element 572 (e.g., a hold capacitor or a rechargeable battery functioning as an internal power storage) that is configured to apply current and potential to the sub-module 550, 552 when temporally disconnected from the main module 510. In some embodiments, sensor data is continuously transmitted (e.g., while connected or while disconnected from main power) through galvanically separated signal transducers 590, 592. In some embodiments, the galvanically separated signal transducers 590, 592 includes opto-isolators capacitors, Hall effect sensors, magneto resistors, RF, transformers, or relays. In another temporal galvanic separation embodiment, the main module 510 includes a single galvanically separated signal transducer 590 that is selectively connected to one of the sub-modules 550, 552 at a time by a switching element that is situated between the signal transducers 590, 592 and the sub-modules 550, 552.

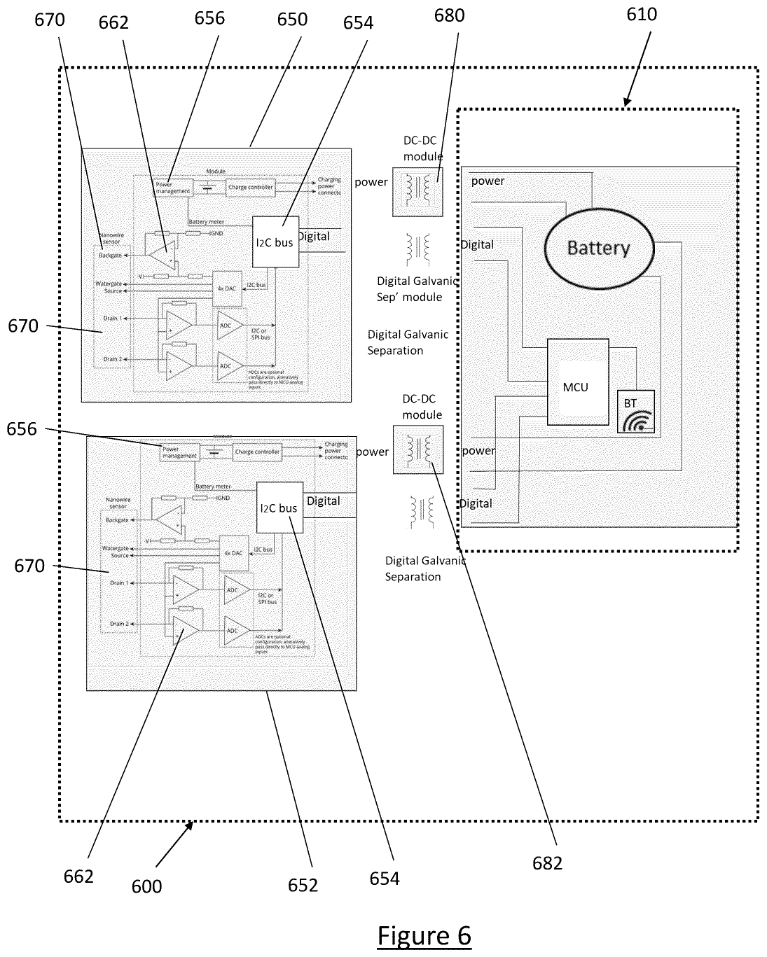

In some embodiments, sensor-specific sub-modules include only an analog “front end” and do not include a microcontroller controller unit. FIG. 6 shows a circuit diagram for a system 600 that is provided in this manner. In some embodiments, the system 600 includes a main module 610 and two sub-modules 650, 652. In some embodiments, each of the sub-modules 650, 652 includes a power management element 656, and one or more amplifiers 662. In some embodiments, each of the sub-modules 650, 652 includes additional electronic hardware elements such as a multiplexer, a memory, etc. In some embodiments, the one or more amplifiers 662 are coupled to a sensor 670 (e.g., having a physically separated sensing element as described above). In some embodiments, rather than including an MCU as included in the sub-modules 450, 452, the sub-modules 650, 652 include an Inter-Integrated Circuit (I2C) bus 654 facilitating communication with the main module 610. In some embodiments, each of the sub-modules 650, 652 is powered by corresponding power supply apparatus 680, 682 that are galvanically separated from one another. In some embodiments, such as shown in FIG. 6, the power supply apparatuses 680, 682 are wireless inductance power supplies. In some embodiments, the sub-modules 650, 652 are connected to the main module 610 under galvanic separation conditions. In some embodiments, the sub-modules 650, 652 are wirelessly connected to the main module 610.

In some embodiments, to further benefit from the separation between digital to analog front-end, some of the front-end function is incorporated directly into the physical sensor chip. For example, in some embodiments, since sensors chips are fabricated using CMOS fabrication and include additional non-functional silicon area, some or all of the analog/digital components are incorporated directly into the sensor silicon-based chip as part of an Application Specific Integrated Circuit (ASIC).

In some embodiments, physical galvanic separation of each sensor chip is performed at the MEMS deep etch step, which directly results in an isolated integrated chip. In some embodiments, the fact that the sensor sub-modules are galvanically separated in-situ and assembled at the wafer level results in highly accurate (e.g., with tolerance of under 1 micron) and repetitive chip-to-chip distance. In some embodiments, chip-to-chip distance of under 20 microns is achieved.

In some embodiments, having a predefined probe-to-probe relative placement, as well as high spatial correlation between the chip's probe and the PCB layer, as a result of a wafer-level assembly, enables highly efficient design and fabrication of wireless power and data communication. Additionally, in some embodiments, having a predefined main module ASIC chip which is located in proximity to the MEMS-isolation etch (e.g., within 50 microns) allows low power Wi-Fi communication as the distances between receiver and transducer are minimized and are accurately predefined.

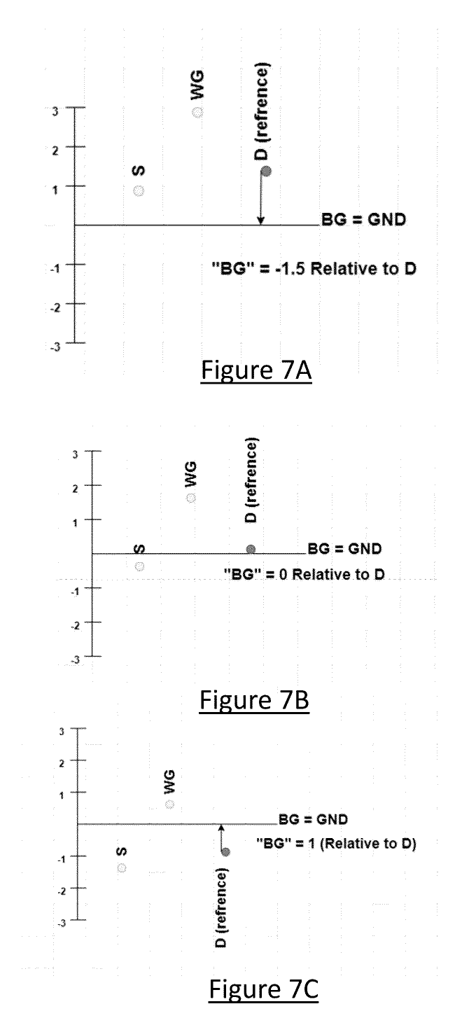

In some embodiments, a virtual floating voltage control is implemented to facilitate integration of a biosensor, such as an electrochemical potentiometric FET based sensor having a variable backgated-biasing, with other application-specific integrated circuit (ASIC) elements. As discussed herein, exemplary field effect transistor-based sensors are implemented for sensing chemical or biological analytes. In such sensors, a backgate voltage can be adjusted to configure the operation of the sensor, to thereby tune the sensor for sensing of different analytes. For example, in some cases, biological and chemical sensors use the bulk of the wafer, sometimes referred to as the “handle”, as a connection point to a voltage source, such that the voltage source can be operated to apply a specific voltage to the backgate at a desired time. However, in some cases, adjustment of backgate voltage in a sensor system with plural sensors in close proximity to one another can cause interference that causes sensor data to be unreliable. In For example, integrating a biological or a chemical sensor with an ASIC, such as a front end analog ASIC or mixed analog/digital ASIC such as those described herein, on a mutual substrate has the effect that any change to the shared handle potential will influence the performance of the ASIC analog/digital transistors, such that their threshold voltage will change, which might result in system instability and failure. Accordingly, in some embodiments, to apply a constant potential or grounding to the handle of such integrated chip (and thereby avoid the accompanying potential instability as described above), while still enabling backgate tuning to tune the performance of a given sensor to sense a desired analyte, a floating virtual voltage control can be implemented. In some embodiments, a floating voltage control includes grounding the backgate (or, in other embodiments, maintaining the backgate at a nonzero fixed voltage), while shifting the potential of other components of the same sensor (e.g., the working electrode, source, and drain potentials) in parallel relative to the handle, to thereby control relative potential of a given backgate. As the sensing performance of the sensor depends on the voltage of the backgate relative to the other components, such shifting tunes the performance of the sensor, while maintaining the backgate itself at a fixed voltage. For example, FIGS. 7A-7C show diagrams illustrating this effect. In FIGS. 7A-7C, the backgate is set at a fixed grounded voltage, while the source voltage S, drain voltage D, and working electrode WG are adjusted relative to the backgate voltage BG and in parallel to one another. In FIG. 7A, the virtual backgate voltage (e.g., voltage of the backgate relative to the drain voltage D, used as a reference point) is −1.5. In FIG. 7B, the virtual backgate voltage is 0. In FIG. 7C, the virtual backgate voltage is 1. As the actual voltage of the BG is not changed, all other on-chip components such as analog/digital components are not affected by such changes change. In some embodiments, virtual voltage control as described herein can be implemented by software control (e.g., by controlling voltage applied by a DAC).

In another embodiment, a chemical or biological FET based sensor includes no virtual floating voltage control, meaning an ungrounded backgate having a variable voltage. This ungrounded backgate system, when integrating with an analog/digital ASIC, has a need for separating the backgate voltage of the sensor FET area (sensor backgate) from the analog/digital ASIC so that each component can be controlled separately while not affecting the other component. In such embodiments, backgate separation can be achieved by: (1) physically separating the wafer handle silicon bulk by dielectrics or etching; (2) separating the electric potential in different parts of the wafer handle by implanting different dopants to achieve N/P junction; (3) adding localized backgate wells (sub-FET dedicated bias backgate) under the other components (e.g., sensor and ASIC); (4) adding localized backgate wells under each transistor on the chip (sensor and ASIC) such that each transistor or transistor array (for example. ADC, DAC, AMP, resistors, memory cells) can be individually controlled; or (5) adding backgate wells above or below the buried oxide layer of a silicon-on-insulator wafer.

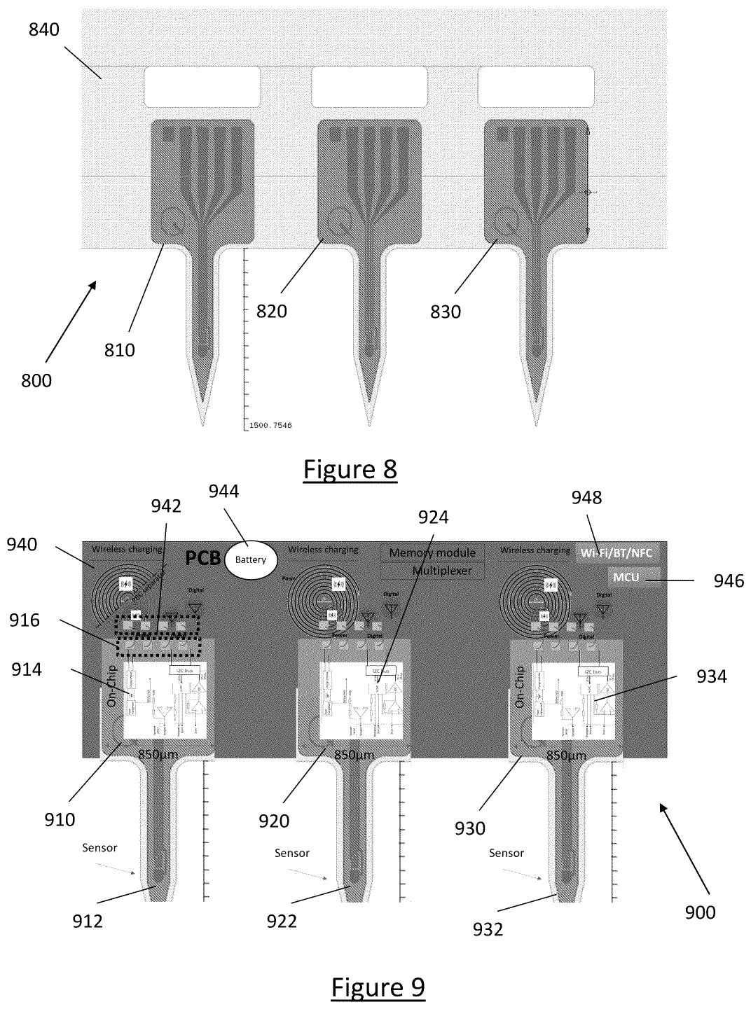

FIG. 8 shows an embodiment of a sensing device 800. In the embodiment shown in FIG. 8, the sensing device 800 includes three sensor probes 810, 820, 830. The sensor probes 810, 820, 830 are physically galvanically separated from one another as described herein. The sensor probes 810, 820, 830 are mounted on a support layer 840. In some embodiments, the sensor probes 810, 820, 830 are mounted on the support layer 840 using a non-dielectric adhesive as described herein. In some embodiments, a PCB layer is positioned “below” the support layer 840 (e.g., on the side of the support layer 840 opposite the sensor probes 810, 820, 830) as described above with reference to FIGS. 2A-2B. In some embodiments, all assemblies are performed at the wafer level scale.

FIG. 9 shows an embodiment of a sensing device 900. In the embodiment shown in FIG. 9, the sensing device 900 includes three sensor probes 910, 920, 930. The sensor probes 910, 920, 930 are physically galvanically separated from one another as described herein. In some embodiments, each of the sensor probes 910, 920, 930 includes a respective sensor 912, 922, 932 that is positioned at the end of the respective sensor probe 910, 920, 920, and is configured to be inserted into an analyte solution or tissue. In some embodiments, each of the sensors 912, 922, 932 is a field effect transistor sensor. The sensor probes 910, 920, 930 include respective on-chip integrated analog front ends 914, 924, 934 such as described above with reference to FIG. 6. In some embodiments, each of analog front ends 914, 924, 934 includes ADC, DAC, voltage control units, and amplifiers. In some embodiments, each of the analog front ends 914, 924, 934 is configured to supply electrical potential to the source, working electrode, and back gate of the corresponding one of the sensors 912, 922, 932, and to collect and amplify electrical current from the field effect transistor drain of the corresponding one of the sensors 912, 922, 932. In some embodiments, communication to and from each of the sensor probes 910, 920, 930 performed using a low output/input data architectures such as Inter-Integrated Circuit (I2C). The sensing device 900 includes a main PCB 940. In some embodiments, each of the probes 910, 920, 930 is connected to the PCB 940 via physical connection, such as wire-bonding. In some embodiments, such as shown in FIG. 9, each of the probes 910, 920, 930 includes four pads 916 (specifically identified only for the probe 910 for clarity) that are coupled to four corresponding pads 942 on the PCB 940. In some embodiments, two of the pads 916 for each of the probes 910, 920, 930 provide power supply and the remaining two of the pads 916 provide data communication. In other embodiments, only two pads connect the PCB 940 to each of the probes 910, 920, 930, and power supply and data communication are provided over the same connections. In some embodiments, each of the sensor probes 910, 920, 930 is galvanically separated at the PCB level via in-situ wireless power transmission, for example by inductive, capacitive electrodynamic magneto dynamic or other ways of wireless power/energy transmission. In some embodiments, the wireless energy transfer is achieved via coil-to-coil induction, which is either added as a discrete component onboard the PCB 940 or integrated into the layers of the fabricated PCB 940 whereas the power source coil and the receiver coil are separated by a PCB insulation layer. In some embodiments, bidirectional data communication is achieved via the same wireless power system or by separate, conductive, capacitance, electrooptical, magnetic or RF transducers. In some embodiments, the PCB 940 includes a battery 944, a microcontroller controller unit 946, and a communication interface 948 enabling communication to a further device. In some embodiments, the communication interface 948 is a wireless communication interface. In some embodiments, the communication interface 948 includes Bluetooth, WiFi, near field communication (NFC), NarrowBand Internet of Things (NB-IoT), long range (LoRa), or the like. In other embodiments, the PCB 940 also includes one or more multiplexers, power management, or memory. In other embodiments, the PCB 940 includes further sensors, such as accelerometers, temperature sensors, electro-optical sensors for pulse oximetry, contact electrodes (e.g., ECG electrodes), or another type of sensor that may be suitable for health monitoring and/or patient treatment prediction. In some embodiments, the analog front end of the probes 910, 920, 930 is made of a separate silicon die assembled on top of the silicon probe chip, such that both are connected electrically in a method such as flip chip or any similar method for die-to-die electrical connection. In some embodiments, a support layer (e.g., a metal support layer) is present between the sensor probes 910, 920, 930 and the PCB 940 and is adhered by an adhesive (e.g., a non-dielectric adhesive) as described above at least with reference to FIG. 3B.

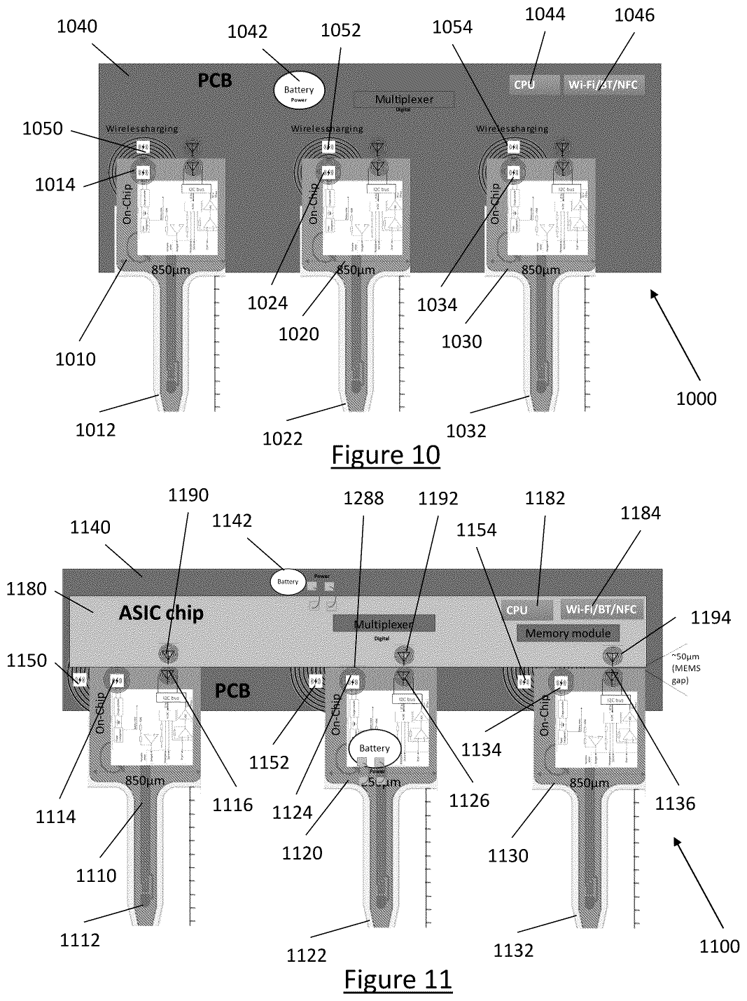

FIG. 10 shows an embodiment of a sensing device 1000. In the embodiment shown in FIG. 10, the sensing device 1000 includes three sensor probes 1010, 1020, 1030. The sensor probes 1010, 1020, 1030 are physically galvanically separated from one another as described herein. In some embodiments, each of the sensor probes 1010, 1020, 1030 includes a respective sensor 1012, 1022, 1032 that is positioned at the end of the respective sensor probe 1010, 1020, 1020, and is configured to be inserted into an analyte solution or tissue. In some embodiments, each of the sensors 1012, 1022, 1032 is a field effect transistor sensor. The sensing device 1000 includes a PCB 1040. In the embodiment of FIG. 10, each of the sensor probes 1010, 1020, 1030 is connected to the PCB 1040 wirelessly and with no wired connections. In some embodiments, the PCB 1040 includes a battery 1042, a microcontroller controller unit 1044, and a communication interface 1046 enabling communication to a further device. In some embodiments, the communication interface 1046 is a wireless communication interface. In some embodiments, the communication interface 1046 includes Bluetooth, WiFi, near field communication (NFC), NarrowBand Internet of Things (NB-IoT), long range (LoRa), or the like. In other embodiments, the PCB 1040 also includes one or more multiplexers, power management, or memory. In some embodiments, each of the sensor probes 1010, 1020, 1030 includes at least one wireless connecting element 1014, 1024, 1034, respectively, and the PCB 1040 includes at least one wireless connecting element 1050, 1052, 1054 corresponding to each of the sensor probes 1010, 1020, 1030. In some embodiments, a first wireless connection between the PCB 1040 and each of the sensor probes 1010, 1020, 1030 provides power transfer (e.g., from the battery 1042), and a second wireless connection between the PCB 1040 and each of the sensor probes 1010, 1020, 1030 provides data transfer. In some embodiments, a first wireless connection between the PCB 1040 and each of the sensor probes 1010, 1020, 1030 provides both power transfer (e.g., from the battery 1042) and data transfer. In some embodiments the analog front end (e.g., of each of the sensor probes 1010, 1020, 1030) is made of a separate silicon die assembled on top of the silicon probe chip, such that both are connected electrically in a method such as flip chip or any similar method for die-to-die electrical connection. In some embodiments, a support layer (e.g., a metal support layer) is present between the sensor probes 1010, 1020, 1030 and the PCB 1040 and is adhered by an adhesive (e.g., a non-dielectric adhesive) as described above at least with reference to FIG. 3B. In some embodiments, the wireless connecting elements 1014, 1024, 1034 and/or the wireless connecting elements 1050, 1052, 1054 are integrated into the sensor probes 1010, 1020, 1030 and/or the PCB 1040 during a CMOS fabrication step (e.g., in order to form silicon-embedded metal coils for energy transfer and/or data transfer from the sensor probes 1010, 1020, 1030 to/from the PCB 1040.

FIG. 11 shows an embodiment of a sensing device 1100. In the embodiment shown in FIG. 11, the sensing device 1100 includes three sensor probes 1110, 1120, 1130. The sensor probes 1110, 1120, 1130 are physically galvanically separated from one another as described herein. In some embodiments, each of the sensor probes 1110, 1120, 1130 includes a respective sensor 1112, 1122, 1132 that is positioned at the end of the respective sensor probe 1110, 1120, 1120, and is configured to be inserted into an analyte solution or tissue. In some embodiments, each of the sensors 1112, 1122, 1132 is a field effect transistor sensor. The sensing device 1100 includes a PCB 1140. In the embodiment of FIG. 11, each of the sensor probes 1110, 1120, 1130 is connected to the PCB 1140 wirelessly and with no wired connections. In some embodiments, the PCB 1140 includes a battery 1142. In the embodiment shown in FIG. 11, the sensing device 1100 includes an application-specific integrated circuit (ASIC) 1180 including a microcontroller controller unit (MCU) 1182 and a communication interface 1184. In some embodiments, the communication interface 1184 is a wireless communication interface. In some embodiments, the communication interface 1184 includes Bluetooth, WiFi, near field communication (NFC), NarrowBand Internet of Things (NB-IoT), long range (LoRa), or the like. In some embodiments, the ASIC 1180 is fabricated out of a silicon frame which is not directly part of the sensor probes 1110, 1120, 1130 or the analog front ends thereof. In some embodiments, each of the sensor probes 1110, 1120, 1130 includes a first wireless connecting element 1114, 1124, 1134, respectively, and a second wireless connecting element 1116, 1126, 1136, respectively. In some embodiments, the PCB 1140 includes wireless charging links 1150, 1152, 1154 that are configured to power the respective sensor probes 1110, 1120, 1130 via the respective first wireless connecting elements 1114, 1124, 1134. In some embodiments, the ASIC 1180 includes wireless data links 1190, 1192, 1194 that are configured to communicate data to and from the respective sensor probes 1110, 1120, 1130 via the respective second wireless connecting elements 1116, 1126, 1136. In some embodiments, as all silicon chips are made from the same wafer and are separated by deep etch singulation, distances between elements such as the second wireless connecting elements 1116, 1126, 1136 and the wireless data links 1190, 1192, 1194 are controlled to improve efficiency of such elements. In some embodiments, the MCU 1182 of the ASIC 1180 is located on a different die, such that the ASIC 1180 can be fabricated using an analog-based fabrication process. In some embodiments, the PCB 1140 also includes one or more multiplexers, power management, or memory. In some embodiments, a support layer (e.g., a metal support layer) is present between the sensor probes 1110, 1020, 1030 and the PCB 1140 and is adhered by an adhesive (e.g., a non-dielectric adhesive) as described above at least with reference to FIG. 3B. In some embodiments, the first wireless connecting elements 1114, 1124, 1134 and/or the second wireless connecting elements 1116, 1126, 1136, and/or the wireless charging links 1150, 1152, 1154, and/or wireless data links 1190, 1192, 1194 integrated into the sensor probes 1110, 1120, 1130, and/or the PCB 1140, and/or the ASIC 1180 during a CMOS fabrication step (e.g., in order to form silicon-embedded metal coils for energy transfer and/or data transfer from the sensor probes 1110, 1120, 1130 to/from the PCB 1140 and/or the ASIC 1180.

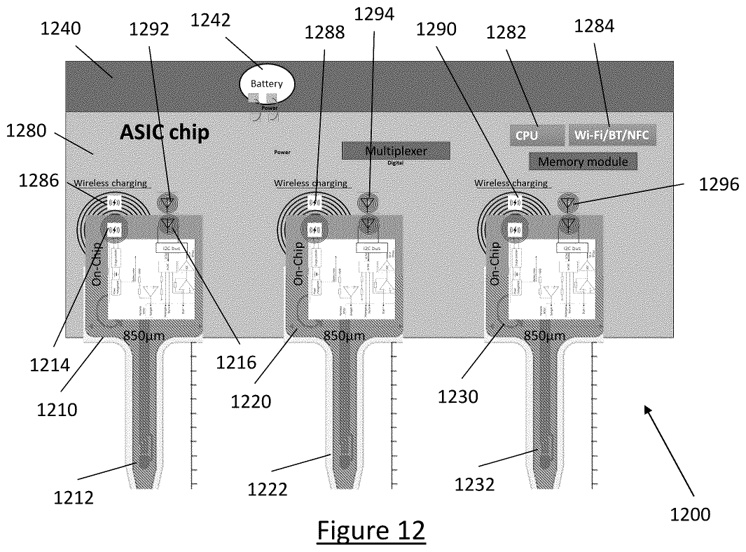

FIG. 12 shows an embodiment of a sensing device 1200. In the embodiment shown in FIG. 12, the sensing device 1200 includes three sensor probes 1210, 1220, 1230. The sensor probes 1210, 1220, 1230 are physically galvanically separated from one another as described herein. In some embodiments, each of the sensor probes 1210, 1220, 1230 includes a respective sensor 1212, 1222, 1232 that is positioned at the end of the respective sensor probe 1210, 1220, 1220, and is configured to be inserted into an analyte solution or tissue. In some embodiments, each of the sensors 1212, 1222, 1232 is a field effect transistor sensor. The sensing device 1200 includes a PCB 1240 and an application-specific integrated circuit (ASIC) 1280. In the embodiment of FIG. 12, each of the sensor probes 1210, 1220, 1230 is connected to the ASIC 1280 wirelessly and with no wired connections. In some embodiments, the PCB 1240 includes a battery 1242 that is coupled to the ASIC 1280. In the embodiment shown in FIG. 12, ASIC 1280 includes a microcontroller controller unit (MCU) 1282 and a communication interface 1284. In some embodiments, the communication interface 1284 is a wireless communication interface. In some embodiments, the communication interface 1284 includes Bluetooth, WiFi, near field communication (NFC), NarrowBand Internet of Things (NB-IoT), long range (LoRa), or the like. In some embodiments, the ASIC is fabricated out of a silicon frame which is not directly part of the sensor probes 1210, 1220, 1230 or the analog front ends thereof. In some embodiments, each of the sensor probes 1210, 1220, 1230 includes a first wireless connecting element 1214 and a second wireless connecting element 1216 (identified with reference numerals only for the sensor probe 1210 for clarity). In some embodiments, the ASIC 1280 includes wireless charging links 1286, 1288, 1290 that are configured to power the respective sensor probes 1210, 1220, 1230 via the respective first wireless connecting elements 1214. In some embodiments, the ASIC 1280 includes wireless data links 1292, 1294, 1296 that are configured to communicate data to and from the respective sensor probes 1210, 1220, 1230 via the respective second wireless connecting elements 1216. In some embodiments, as all silicon chips are made from the same wafer and are separated by deep etch singulation, distances between elements such as the second wireless connecting elements 1216 and the wireless data links 1290, 1292, 1294 are controlled to improve efficiency of such elements. In some embodiments, the MCU 1282 of the ASIC 1280 is located on a different die, such that the ASIC 1280 can be fabricated using an analog-based fabrication process. In some embodiments, the PCB 1240 also includes one or more multiplexers, power management, or memory. In some embodiments, the ASIC 1280 and the sensor probes 1210, 1220, 1230 are connected by an insulating adhesive such that all power and communication among different modules (e.g., between and among the sensor probes 1210, 1220, 1230, and the ASIC 1280) is conducted wirelessly. In some embodiments, a support layer (e.g., a metal support layer) is present between the sensor probes 1210, 1220, 1230 and the ASIC 1280 and is adhered by an adhesive (e.g., a non-dielectric adhesive) as described above at least with reference to FIG. 3B. In some embodiments, the first wireless connecting elements 1214 and/or the second wireless connecting elements 1216, and/or the wireless charging links 1286, 1288, 1290, and/or the wireless data links 1292, 1294, 1296 are integrated into the sensor probes 1210, 1220, 1230 and/or the ASIC 1280 during a CMOS fabrication step (e.g., in order to form silicon-embedded metal coils for energy transfer and/or data transfer from the sensor probes 1210, 1220, 1230 to/from the ASIC 1280.

In any of the embodiments described herein, a sensing device (such as the PCB of a sensing device) may include further sensors. In some embodiments, the further sensors may include accelerometers, temperature sensors, electro-optical sensors for pulse oximetry, and/or contact electrodes (e.g., ECG electrodes). In some embodiments, the further sensors suitable for health monitoring and/or patient treatment prediction.

Various exemplary embodiments have been described herein and depicted in the accompanying drawings that include certain specific quantities of sensors (and corresponding sensor-specific accompanying hardware), such as two sensors, three sensors, etc. Any such quantities described throughout this disclosure are only exemplary and other embodiments may include any other quantity of sensors without departing from the general principles set out herein with reference to the exemplary embodiments.

It is appreciated that certain features of the disclosure, which are, for clarity, described in the context of separate embodiments, may also be provided in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not considered essential features of these embodiments, unless the embodiment is inoperative without those elements.

What is claimed is:

1. A sensor system, comprising:
a plurality of potentiometric sensors,
wherein each of the potentiometric sensors has a sensor area of less than 1 square centimeter,
wherein each of the potentiometric sensors comprises one working electrode, and
wherein each one of the potentiometric sensors is galvanically separated from all other ones of the potentiometric sensors so as to avoid measurement crosstalk among the plurality of potentiometric sensors;
a plurality of sensor control subunits,
wherein each of the sensor control subunits is communicatively coupled to a particular corresponding one of the plurality of potentiometric sensors, and
wherein each one of the sensor control subunits is galvanically separated from all other ones of the sensor control subunits so as to avoid measurement crosstalk among the plurality of sensor control subunits; and
a main control unit communicatively coupled to each of the plurality of sensor control subunits.

2. The sensor system of claim 1, wherein the potentiometric sensors and the sensor control subunits are galvanically separated from one another by physical separation that no electrical or dielectric contact is present between the potentiometric sensors and the sensor control subunits.

3. The sensor system of claim 2, wherein the physical separation is achieved by physical bulk separation at wafer level.

4. The sensor system of claim 2, wherein the physical separation is achieved by a dielectric or polymeric material.

5. The sensor system of claim 1, wherein the main control unit and the plurality of sensor control subunits are galvanically separated from one another through use of wireless or capacitance power transmission.

6. The sensor system of claim 1, wherein the main control unit and the plurality of sensor control subunits communicate with one another while galvanically separated by wireless, capacitance, optical or RF data signal transfer

7. The sensor system of claim, 1 wherein the main control unit is connected to the plurality of sensor control subunits by a switch that is configured to provide active connection between the main control unit and only one of the plurality of sensor control subunits at any point in time.

8. The sensor system of claim 7, wherein each of the plurality of sensor control subunits comprises an internal power storage, and wherein the internal power storage powers the sensor control subunits when not actively connected to the main control unit.

9. The sensor system of claim, 1 wherein the main control unit and each of the sensor control subunits each comprise a microcontroller.

10. The sensor system of claim, 1 wherein the main control unit comprises a microcontroller, and wherein each of the sensor control subunits does not comprise a microcontroller.

11. The sensor system of claim 10, wherein each of the sensor control subunits comprises an analog front end.

12. The sensor system of claim 11, wherein the analog front end comprises an application-specific integrated circuit.

13. The sensor system of claim 1, wherein the main control unit and the plurality of sensor control subunits are implemented on a printed circuit board.

14. The sensor system of claim 13, wherein a support layer is positioned between (1) the printed circuit board and (2) the main control unit and the plurality of sensor control subunits.

15. The sensor system of claim 14, wherein the main control unit and the plurality of sensor control subunits are fixed to the support layer by a non-dielectric adhesive.

16. The sensor system of claim 1, wherein each of the potentiometric sensors comprises only one working electrode.

17. The sensor system of claim 1, wherein each of the potentiometric sensors comprises no reference electrode.
